(19) United States
(12) Patent Application Publication
(10) Pub. No.: US 2017/0014605 A1
(43) Pub. Date: Jan. 19, 2017

(54) IMPROVED BALLOON CATHETER WITH PEELABLE REMOVABLE SHEATH FOR USE IN INDUCING LABOR

(71) Applicant: Barbara SCHULZ, Manhattan Beach, CA (US)

(72) Inventor: Barbara SCHULZ, Manhattan Beach, CA (US)

(21) Appl. No.: 15/123,976

(22) PCT Filed: Mar. 4, 2015
(86) PCT No.: PCT/US15/18591

§ 371 Date: Sep. 6, 2016

Related U.S. Application Data
(60) Provisional application No. 61/948,988, filed on Mar. 6, 2014.

(51) Int. Cl. A61M 29/02; A61B 17/42; A61M 25/06

(52) U.S. Cl. CPC A61M 29/02; A61M 25/0668; A61B 17/42; A61M 2025/0681

(57) ABSTRACT

The claimed invention provides a catheter for inducing labor comprising a balloon catheter; a removable sheath (preferably peelable) that covers the inflation tube; wherein the sheath is removable after insertion of the catheter by removing/peeling off the sheath from the inflation tube.

### CROSS-REFERENCE TO RELATED APPLICATIONS

This application claims priority to U.S. Provisional Patent Application No. 61/948,988, filed on Mar. 6, 2014, which is hereby incorporated by reference in its entirety.

### BACKGROUND OF THE INVENTION

Cervical ripening, or opening of the cervix prior to the onset of labor, is under hormonal control. Failure of the cervix to ripen at term increases the chance of delivery problems, including the need for cesarean section. Further consequences include prolonged hospital stays, increased medical costs, and an overall increase in maternal and fetal morbidity. There are several devices and methods presently utilized to induce artificially the ripening of the cervix to prevent pregnancy complications. One of the most widely used methods involves the intravenous administration of oxytocin. This method, however, causes a prolonged period of induction, tends to suffer from a large failure rate, and may cause patient discomfort. Other common methods of inducing labor include administration of prostaglandin (PG), either systemically or by topical application. Like oxytocin, PG may cause certain undesirable side effects, such as nausea, vomiting, and uterine hypertoxicity.

Mechanical methods of inducing ripening include the use of balloon catheters and hygroscopic cervical dilators. Such devices have the advantage of causing a gradual cervical dilation and minimizing patient discomfort. Several types of catheters have been utilized for this purpose. One study used a transcervical Foley catheter for inducing labor. This procedure was effective in inducing labor and has the benefit of being an inexpensive procedure. This procedure seems to work well, but the catheter is flimsy and is often difficult to place trans-vaginally into the cervix, especially in large/heavy patients.

Another example is that disclosed in U.S. Pat. No. 4,976,692 to Atad et al., showing a two balloon catheter that is placed such that one balloon is in the uterus, and the other balloon is in the vagina. Each balloon connects separately to flexible tubing, permitting independent filling or emptying of each balloon. This particular catheter also has a third flexible tube that allows injection of pharmaceutical agents through a gap between both balloons, or inside the cervix. However, this catheter requires manipulation and adjustment of three different valves arranged in a cumbersome fashion, and is considerably more expensive. Moreover, the administration of pharmaceutical agents in the cervical area through this catheter may fail to deliver the entire dose, as the agents must travel from the valve through the tube to the discharge site.

Thus, there remains a need for a simple yet effective product to use in the ripening of the cervix and inducement of labor.

### SUMMARY OF THE INVENTION

The claimed invention provides a catheter for inducing labor comprising a balloon catheter; a removable sheath (preferably peelable) that covers the inflation tube; wherein the sheath is removable after insertion of the catheter by peeling off the sheath from the inflation tube.

The catheter may be any suitable balloon catheter. In certain embodiments, the catheter is a Foley catheter. In certain embodiments, the catheter is a 14, 16 or 18 French sized catheter, with either a 30, 60 or 80 ml balloon.

The sheath may comprise perforations and or tabs or flaps to facilitate the peeling off of the sheath after insertion.

### BRIEF DESCRIPTION OF THE DRAWINGS

FIG. 1 shows one form of the catheter constructed in accordance with the present invention. FIG. 1A shows the balloon uninflated and FIG. 1B shows the balloon inflated.

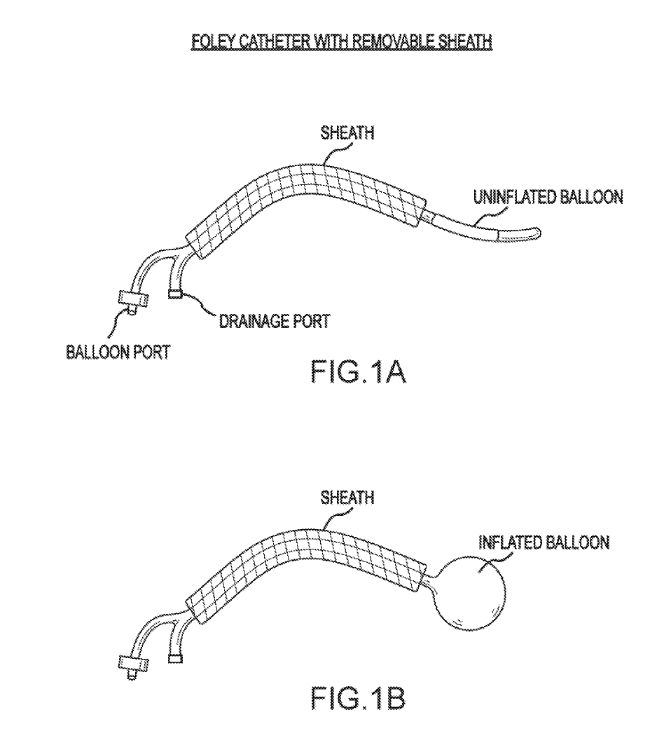

FIG. 2 shows the sheath with perforations and/or tabs. FIG. 2A shows the sheath with perforations at the ends whereas FIG. 2B shows the perforations running the entire length of the sheath. FIGS. 2C and 2D show the sheath with a tab.

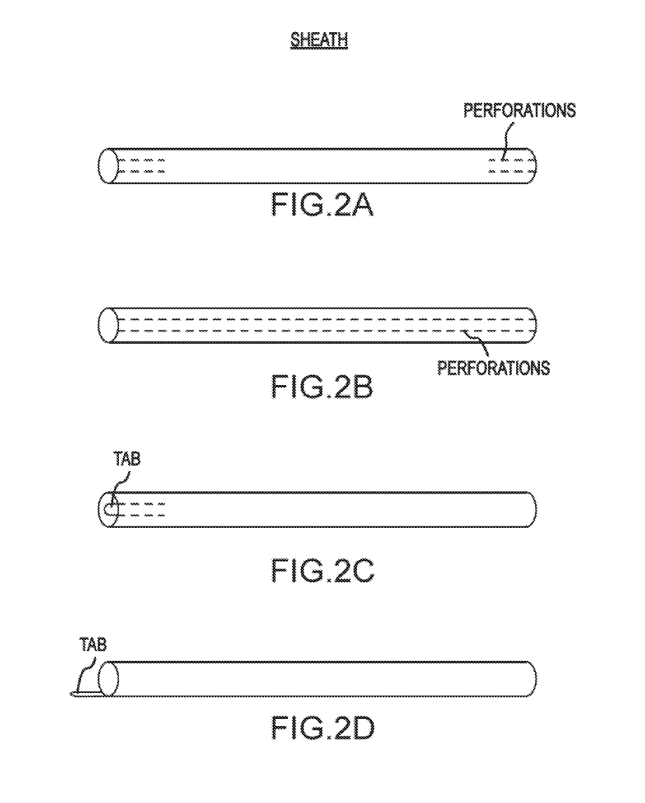

FIG. 3 depicts the sheath being peeled away from the catheter filling tube.

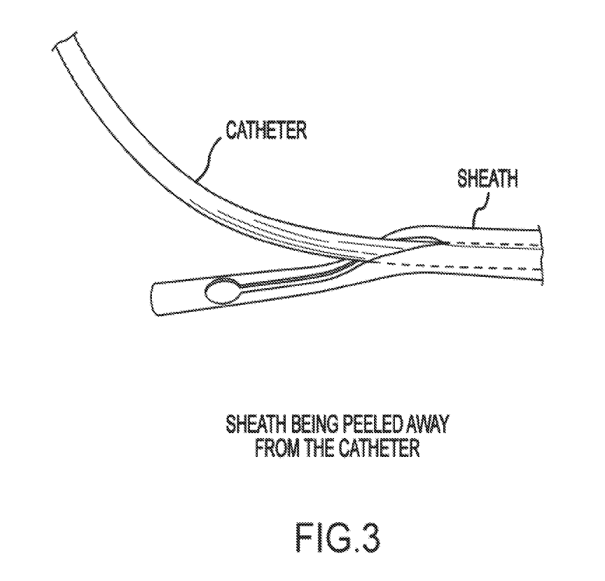

### DETAILED DESCRIPTION OF THE INVENTION

The claimed invention provides an improved single balloon catheter useful to induce cervical ripening. The balloon catheter has a removable sheath surrounding the inflation tube. The sheath provides rigidity and strength to the tube in aid in the insertion but it is removable by tearing or ripping away of the sheath after insertion.

The goal of the cervical ripening/induction is to cause the cervix to mechanically open. Sometimes this will start labor spontaneously and sometimes it will simply make the cervix more favorable for an application of a drug, such as Pitocin, or for amniotomy.

The catheter can be any flexible balloon catheter and is preferably a Foley type catheter or similar catheter. A Foley catheter is a soft, rubber catheter that is normally used by caregivers to empty the bladder of urine. When the catheter is inserted into the bladder, a small balloon in the end of the tube is inflated with sterile water or saline, to stop the tube from falling out. When the Foley catheter is used to induce labor, the tip is placed into the opening of the cervix, and the balloon is gently inflated. This puts even pressure on the inside of the woman's cervix. The pressure is aimed at releasing local prostaglandins (that can ripen the cervix) and/or dilate the cervix to about 3 to 4 cms. In some cases the catheter will induce the labor contractions. However, often the waters need to be broken by the caregiver at some stage, for the labor to keep progressing until the actual birth.

The catheter comprises an external removable sheath that envelopes the inflation tube/lumen. In some embodiments the sheath covers all or a portion of the uninflated balloon portion of the catheter. In some embodiments, the sheath does not extend to cover the balloon portion of the catheter. After insertion of the catheter into the cervix, the removable sheath is removed. The sheath can be removed before inflation of the balloon. The sheath can also be removed after inflation of the balloon, but in this embodiment, the sheath preferably does not extend to cover the balloon portion of the catheter. The sheath provides stability and rigidity to the catheter during insertion. The sheath may have perforations and/or tab(s) or other means for allowing the sheath to be peeled/torn off rather than slipping off or rolling off. The perforations may extend the entire length of the sheath to aid in the ripping off of the sheath or the perforations may only extend along a small portion of its length to aid in the beginning of the ripping or tearing away. The perforations can be on either end, near the balloon or the distal end, or on both ends. The sheath may have one row of perforations or may have more than one row. The sheath may also have tab(s) or flap(s) to aid in grasping a section to start the tearing away of the sheath. There may be one tab or may be more than one tab. The tabs can be located at either end or may be located at both ends. There may even be tabs located along the length of the sheath at various position or in just one position. In certain embodiments, the tab(s) or flap(s) is located near the perforations. The sheath is made of a material that provides support and rigidity to the catheter tube when present, but at the same time be amendable to ripping or tearing away. The sheath may be tearable/ripable/removable without the need for perforations. An exemplary material is SensaFlex™, which is currently used on Utah Medical Products Inc. IUP Intran®-plus catheter. SensaFlex™ is a tactile tubing material that is stiff enough to allow insertion while maintaining the pliability necessary to avoid injuries. The sheath can be any size necessary to encompass the catheter filling tube and preferably is sized to be snug against the tube and sized not so large as to unduly increase the diameter of the catheter/sheath combination (which could lead to patient discomfort). The sheath is sized thick enough for the particular material used to construct the sheath to provide the necessary support and rigidity. However, the sheath should not so rigid as to prevent ease of insertion and to cause patient discomfort or injury. Further, if too thick, the sheath would not lend itself well to ripping or tearing away from the catheter filling tub. When the sheath is comprised of SensaFlex™, it is preferably about 1½ mm thick or less. In certain embodiment the sheath is about 2 mm thick or less, or ranges from 0.5 mm to 2 mm, or 0.25 mm to 2.5 mm or 0.5 mm to 1.75 mm. or 0.5 mm to 1.5 mm.

The balloon on the catheter can be any shape or size deemed appropriate or necessary to induce labor. For example, the balloon maybe round or pear shape or oval shape or any other desired shape. The catheter may have one or two balloons (as in the case of the Cooks type double balloon catheter). However, a single balloon catheter is preferred, as it is less expensive and less cumbersome to manipulate. In certain embodiments, the balloon is about sized to hold about 30 cc to about 80 cc of fluid or air. In certain embodiments the balloon is sized to hold at least 30 cc, or sized to hold between 40 cc to 60 cc fluid, or sized to hold between 60 to 80 cc fluid.

The balloon, inflation tube and sheath are preferably constructed of biocompatible materials. Biocompatible materials for the balloon should be elastomeric in nature to allow expansion and contraction. These materials include, but are not limited to, latex, rubber including nitrile rubber, styrene butadiene rubber, viton, or butyl rubber, silicone, dacron-reinforced silicon material, polyethylene, Mylar, or Teflon. The filling tube/lumen may be constructed of a like material.

In preferred embodiments the catheter is as Foley-type catheter that is sized as a 14, 16 or 18 French catheter.

The present invention also provides methods of inducing labor by inserting a catheter of the invention into a patient prior to inflation. A portion of the balloon is placed through the undilated or partially dilated cervix such that the distal end of the balloon extends into the uterine region, the proximal end with the inflation tube attached extends into the vaginal region, while the center portion passes into and remains inside the cervical region. Upon inflation with a fluid such as air, sterile water, or a sterile saline solution, the outside wall of the central portion presses radially against the cervix to induce ripening. The sheath is removed after insertion of the catheter. The sheath can be removed before the balloon is inflated or after it is inflated.

The catheter is usually used with gentle traction such as by taping the distal tip of the catheter to the mid thigh. To maintain traction, the distal tip is periodically repositioned on the thigh as necessary. Sometimes other methods are used to apply pressure to the catheter. Sometimes this involves weights, like a liter of fluids or even pulling or tugging on the catheter 2-4 times per hour.

In certain embodiments, the sheath is not removed and is designed to remain on the catheter. In these embodiments, the sheath material does not need to be (but could be) made of a peelable or ripable material.

1. A catheter for inducing labor, the catheter comprising:
a) a balloon catheter comprising an inflation tube and a balloon;
b) a removable sheath that covers the inflation tube; wherein the sheath is removable after insertion of the catheter by peeling off or removing the sheath from the inflation tube.

2. The catheter for claim 1 wherein the catheter is a Foley catheter.

3. The catheter of claim 2 wherein the catheter is a 14, 16 or 18 French sized Foley catheter.

4. The catheter of claim 1 wherein the sheath comprises perforations to facilitate the peeling off of the sheath after insertion.

5. The catheter of claim 4 wherein the sheath further comprises a tab near the perforations to facilitate the peeling off of the sheath after insertion.

6. The catheter of claim 1 wherein the removable sheath is peelable.

7. A method of inducing ripening of the cervix to induce labor using the catheter of claim 1, the method comprising
a) prior to inflation, placing at least a portion of the balloon through an undilated or partially dilated cervix such that a distal end of the balloon extends into the uterine region, and a proximal end with the inflation tube attached extends into the vaginal region, while the center portion of the balloon passes into and remains inside the cervical region;
b) inflating the balloon with a fluid such as air, sterile water, or a sterile saline solution, to cause the outside wall of the balloon to press radially against the cervix to induce ripening of the cervix;
c) removing the sheath after insertion of the catheter.

8. A catheter for inducing labor, the catheter comprising:
a) a balloon catheter comprising an inflation tube and a balloon; and
b) a sheath that covers the inflation tube.
